(19) United States
(12) Patent Application Publication
(10) Pub. No.: US 2025/0281232 A1
(43) Pub. Date: Sep. 11, 2025

(54) HEART VALVE LEAFLET MODIFICATION

(71) Applicant: AMX Technologies, LLC, Plymouth, MN (US)

(72) Inventors: Paul Sorajja, Plymouth, MN (US); Daniel P. Coyle, Plymouth, MN (US); David M. Costello, Plymouth, MN (US)

(73) Assignee: AMX Technologies, LLC, Plymouth, MN (US)

(21) Appl. No.: 18/859,956

(22) PCT Filed: May 5, 2023
(86) PCT No.: PCT/US2023/066712

§ 371 Date: Oct. 24, 2024

Related U.S. Application Data
(60) Provisional application No. 63/364,263, filed on May 5, 2022.

(51) Int. Cl. A61B 18/14; A61B 18/00

(52) U.S. Cl. CPC A61B 18/1492; A61B 2018/0016; A61B 2018/00184; A61B 2018/00369; A61B 2018/00601; A61B 2018/1407

(57) ABSTRACT

A system and method modifying a heart valve leaflet utilizing a leaflet crossing tool and a cutting element.

### RELATED APPLICATIONS

This application claims benefit of and priority to U.S. Provisional Application Ser. No. 63/364,263 filed May 5, 2022 entitled Heart Valve Leaflet Modification, which is hereby incorporated herein by reference in its entirety.

### FIELD OF THE INVENTION

The present disclosure relates to novel and advantageous transcatheter-delivered valve repair devices. More specifically, the devices herein address issues related to treatment of pathology involving the heart valves, such as the mitral, aortic, pulmonary, and tricuspid valves.

### BACKGROUND OF THE INVENTION

The background description provided is for the purpose of generally presenting the context of the disclosure. Work of the presently named inventor(s), to the extent it is described in this background section, as well as aspects of the description that may not otherwise qualify as prior art at the time of filing, are neither expressly nor impliedly admitted as prior art against the present disclosure.

One common cardiovascular pathology is valvular heart disease, which may involve narrowing (i.e., stenosis), incompetence (i.e., insufficiency or regurgitation), or a combination of these two diseases affecting the heart valves in a patient. When valvular heart disease occurs, the chambers of the heart can adversely remodel, leading to heart failure, severe morbidity, and impaired survival.

Open surgery is commonly performed to address such conditions but transcatheter therapy with repair or replacement of the valve often is preferred. The minimally invasive approach offered by transcatheter therapy for treatment minimizes peri-operative and post-operative recovery.

Transcatheter approaches to treat valvular heart disease involve repairing the valve or performing a complete replacement, whereby a prosthesis is implanted inside the patient's native valve. In other cases, a prosthesis may be implanted within a previously placed prosthesis that has become dysfunctional or dislodged (e.g., valve-in-valve).

Transcatheter therapies for valvular heart disease are commonly performed with the native leaflets or prosthetic material intact. Unfortunately, the native leaflets or prosthetic material that is left in place can interfere with the success of subsequent repair or replacement. In some instances, the risk of interference is too high and successful transcatheter therapy is not possible.

One specific example is transcatheter mitral valve replacement, in which a valvular prosthesis is implanted in either a patient's native mitral valve apparatus or a previously placed prosthesis. Due to the proximity of the mitral valve annulus to the left ventricular outflow tract, transcatheter mitral valve replacement may lead to positioning of the native or prosthetic leaflets in the direction of systolic flow and cause obstruction.

Another specific example is transcatheter aortic valve replacement, in which a valvular prosthesis is implanted in either a patient's native aortic valve apparatus or a previously placed prosthesis. Due to the proximity of the aortic valve annulus to the coronary arteries, transcatheter aortic valve replacement may lead to displacement of the native or prosthetic leaflets near the origins of the coronary arteries and impair coronary flow. Such coronary flow impairment can lead to myocardial infarction, ischemia, and death.

In yet another example. it is desirable to modify or remove the native or prosthetic valve leaflets in order to perform valve repair or replacement. These situations can arise because of severe degeneration of the leaflets (e.g., bulky calcification). Modification or removal of such leaflets can improve safety of subsequent valve implantation, as well increase the effectiveness of the valve implantation by creating more room for the prosthesis (i.e., an increase in effective orifice area).

Due to the challenges for open heart surgery procedures and preference for transcatheter-delivered therapies, catheter-based means for modification and/or removal of native and prosthetic leaflets is needed.

### SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure relates to systems and methods for modifying and removing native and prosthetic valve leaflets. The disclosure consists of a delivery catheter (DC), leaflet crossing tool (LCT), and cutting element (CE). Each part or the whole device contains biocompatible material. In at least one embodiment, the DC is steerable and fits inside another steerable guide catheter (SGC). In at least one embodiment, the LCT is first placed across the valve leaflet to determine the site and amount of the cut to be performed, followed by engagement with the CE. The CE, which contemplates sizes and shapes to suit the tissue targeted for cutting, is used to then cut the valve leaflet. In at least one embodiment, the CE and LCT act as a closed loop to capture the cut leaflet tissue for its removal. In at least one alternative embodiment, the cut tissue is removed by mechanical suction through the SGC or DC.

While multiple embodiments are disclosed, still other embodiments of the present disclosure will become apparent to those skilled in the art from the following detailed description, which shows and describes illustrative embodiments. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the disclosure will be better understood from the following description taken in conjunction with the accompanying Figures, in which:

FIG. 1 is a cross-sectional view of the heart, showing the aortic valve (AV), mitral valve (MV), left ventricular outflow tract (LVOT), anterior mitral leaflet (AML), posterior mitral leaflet (PML), left ventricle (LV), and left atrium (LA). The anterior (A) and posterior (P) portions of the MV are marked;

FIGS. 2A-2B are top views of a MV, showing the AML. PML, free edge of the leaflets (FE). The A, P, medial (Med), and lateral (Lat) portions are marked. FIG. 2A is a cross-sectional view in systole; FIG. 2B is a cross-sectional view in diastole;

FIG. 3 shows one embodiment of the LCT and CE, delivered within a DC and SGC. The CE is shown as a loop configuration, which can be enlarged or changed in shape to fit the desired amount and location of the tissue to be removed;

FIGS. 4A-4H show various embodiments of the LCT that can be used to cross and snare the cut tissue;

FIGS. 5A-5F show the technique for use of the present disclosure for modifying and removing leaflet tissue involving the MV. Shown are the aorta, left atrium (LA), MV, and left ventricle (LV);

FIGS. 5G-5L show embodiments for modifying and removing leaflet tissue;

FIGS. 6A-6K show the MV following use of the present disclosure in several embodiments indicating a portion of the valve leaflet removed with a new leaflet edge (NE) created;

FIGS. 7A-7F are cross-sectional views of the heart that focuses on the AV;

FIGS. 8A-8H shows the AV following use of the present disclosure showing the left (L), right (R), and non-coronary (N) cusps of the AV as well the portion of the valve leaflet removed with a new leaflet edge (NE) created;

FIGS. 9A-9H show various embodiments of the CE that can be used to cut the leaflet tissue. Cutting elements on the CE are indicated by the dots;

FIG. 10 shows an embodiment of a LCT;

FIG. 11 shows an embodiment of a CE;

FIG. 12 shows a rendering of a mitral valve;

FIGS. 13A-13F show method of modifying a leaflet of a mitral valve;

FIG. 14 show an element of a method used to modify a leaflet of a mitral valve;

FIG. 15 shows an element of a method used to modify a leaflet of a mitral valve;

FIG. 16 shows an element of a method used to modify a leaflet of a mitral valve; and,

FIG. 17 shows a leaflet modification.

### DETAILED DESCRIPTION

The present disclosure relates to treatment of pathology in the aortic root and ascending aorta using devices deployed via a catheter. Although the present disclosure discusses the embodiments herein with respect to a patient's aortic root and ascending aorta, the embodiments are applicable to any valve of the patient's heart and the disclosure herein must not be construed as to being limited to this application. The embodiments described herein may be applicable to repair of other valves and chambers of the human heart.

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

Heart valve leaflets can be native or prosthetic. The modification and/or removal of heart valve leaflets can be performed to directly repair the function of the valve, or to facilitate its subsequent repair by other means or be treated with valve replacement. The modification and/or removal of the heart valve leaflets needs to be tailored to the patient and the subsequent therapy when indicated.

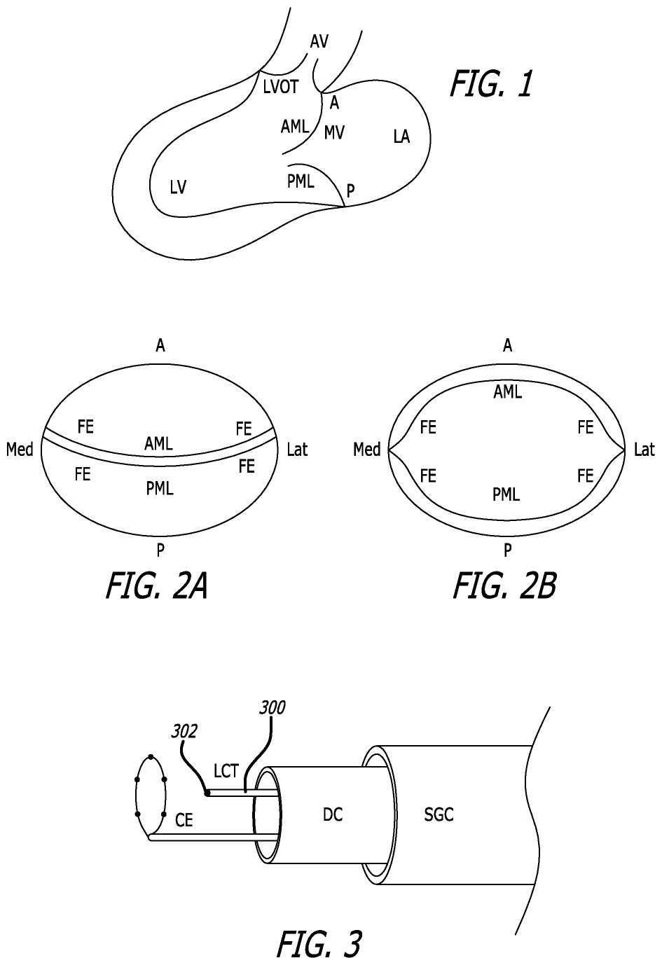

Referring to FIGS. 1 and 2A-2B, the MV typically consists of two leaflets, an AML and PML with FE of both of the leaflets where coaptation is created for valve function in systole and diastole.

When Transcatheter MV Replacement (TMVR) is performed. the AML is typically left intact. The residual presence of the AML encircles the new valve frame, which protrudes into the native LVOT. Obstruction of LVOT flow with TMVR can occur in varying situations, such as if the native LVOT is relatively small in size, if it is set in an unfavorable angle relative to the MV, or if the TMVR prosthesis frame has a configuration that protrudes into the LVOT.

In such scenarios, the protrusion of the new valve frame with an encircled AML can obstruct systolic flow of the heart, leading to impaired stroke volume, low cardiac output, and possible death. The possibility of LVOT obstruction with TMVR is predicted from pre-procedural imaging with cardiac computed tomography or echocardiography. Patients with a significant likelihood of LVOT obstruction with TMVR are not typically allowed to undergo TMVR implantation because of the peri-operative risk.

Cutting and modification of the AML with off-label use of catheters, snares, and wires has been described (also known as a “LAMPOON” procedure). The procedure is limited by relative lack of control of the location of the cut, its amount, and the inability to remove leaflet tissue.

Herein, a tool and its methods are described for accurate and precise cutting and removal of MV leaflets to minimize risk of LVOT obstruction with TMVR, thereby allowing more patients to receive this beneficial therapy for treatment of mitral valve disease.

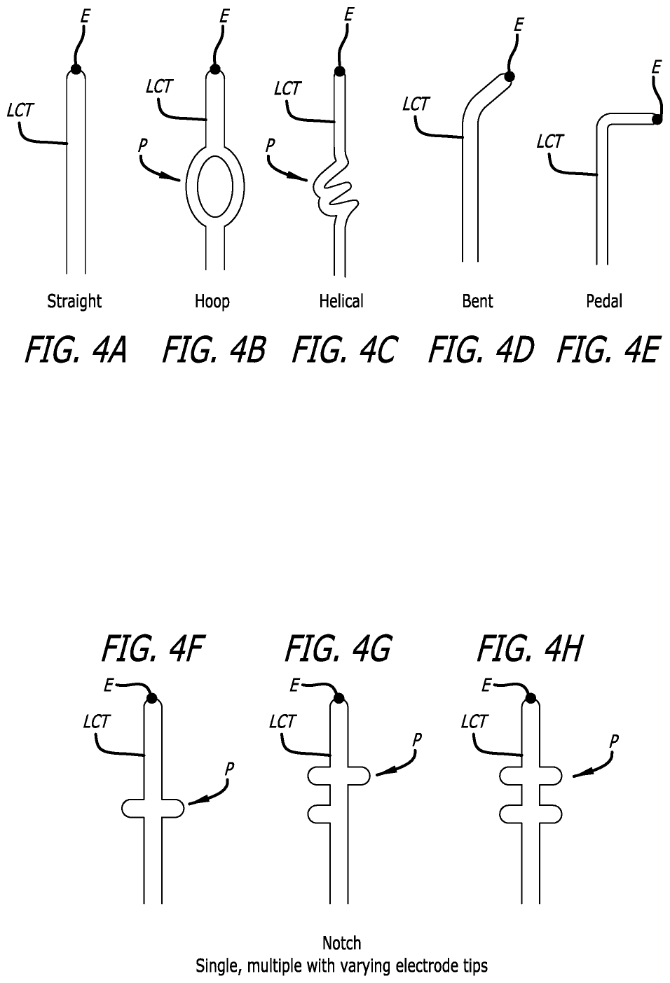

FIG. 3 shows at least one embodiment of having an LCT and CE, that is delivered via a DC housed inside an SGC. The LCT consists of a needle or straight segment 300, with a crossing element 302 at its distal tip whose function is facilitated by mechanical and/or electrical energy. The LCT is used at the targeted site of the valve leaflet for leaflet crossing. As shown in FIG. 4A, in at least one embodiment, the LCT is a straight element.

As shown in FIGS. 4B-4H, in other embodiments, the LCT may be helical, square or rectangular, round or oval, bent, or another geometric configuration, along with a proximal portion P that can be pre-shaped into foldable configurations and used as as an adhesion mechanism for adhering tissue. In at least one embodiment, the proximal portion P is exposed after leaflet crossing. In at least one embodiment, the proximal portion P can be folded to have an anchoring function to hold the leaflet tissue after it is cut. In at least one embodiment, the LCT contains one or multiple elements E that allow delivery of energy to cut the leaflet tissue.

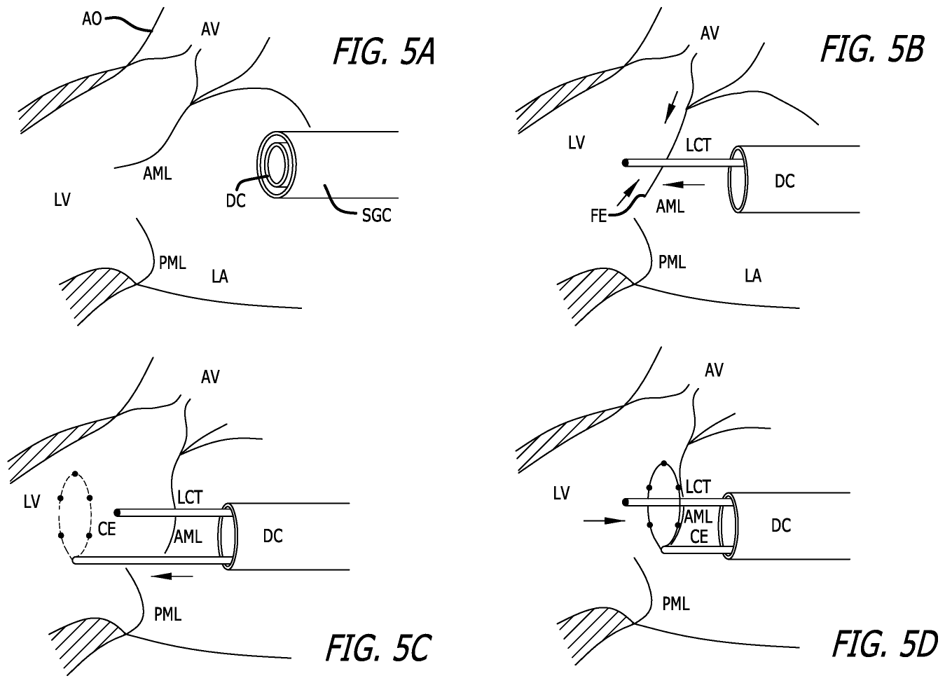

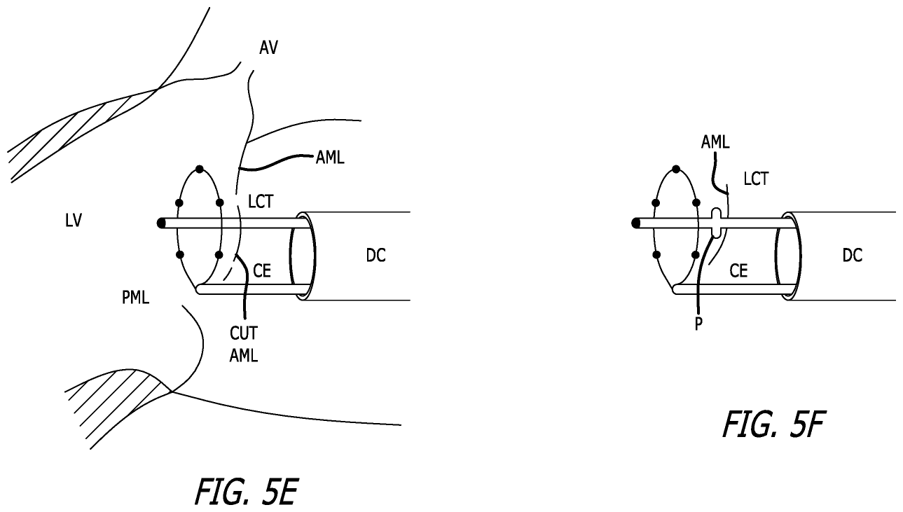

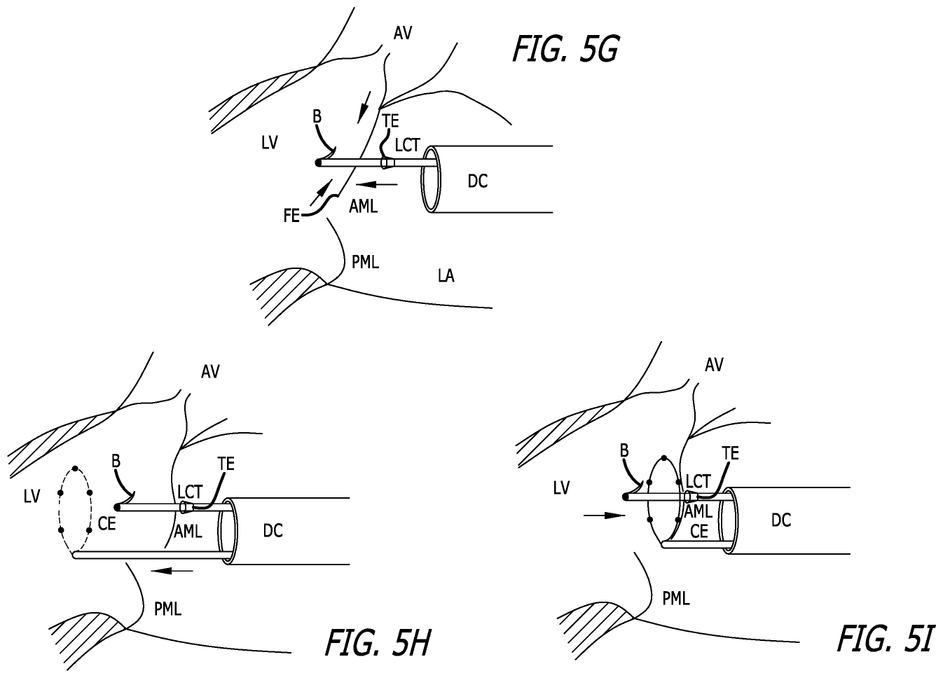

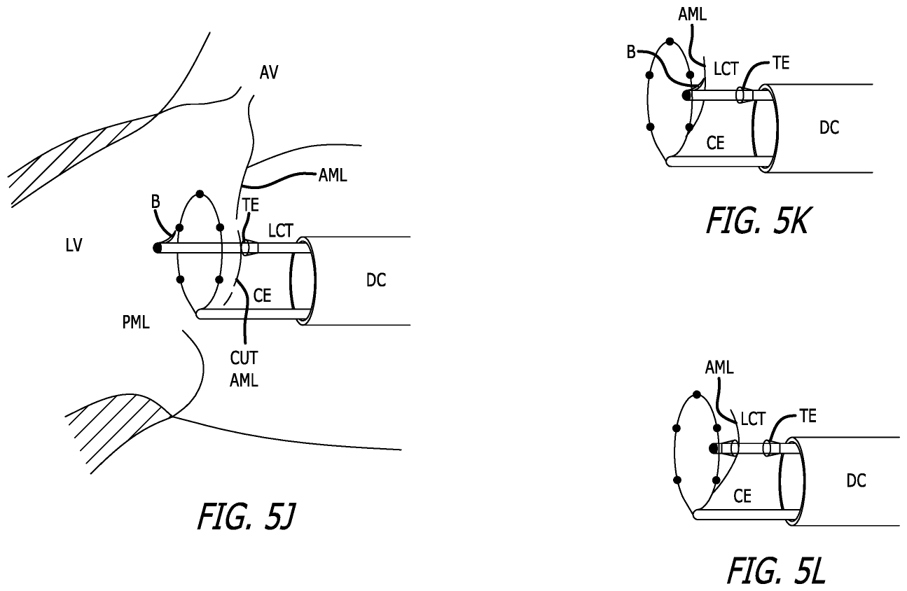

FIGS. 5A-5F show the modification and/or removal of leaflet tissue involving the MV in at least one embodiment of the present disclosure.

Referring to FIG. 5A, the system is delivered to the desired leaflet site with the SGC and the DC is positioned to be adjacent the AML.

Referring to FIG. 5B, the SGC has been withdrawn thereby exposing the DC. The LCT is then advanced out of the DC. The LCT is then used to engage and cross the AML by, for example, radiofrequency, electrical, or mechanical energy, followed by its advancement further into the LV.

The location chosen for placement and engagement of the LCT is based on the location and the amount of leaflet tissue cutting and/or removal that is desired, as indicated from the bidirectional arrows. For example, one may choose to target relatively more tissue by positioning the LCT close to the valve annulus, or relatively less tissue by positioning the LCT close to the free edge (FE) of the leaflet. The CE can also be positioned to cross any segment of the MV as close to the FE or the MV annulus as is desired.

Referring to FIGS. 5C, the CE is then advanced through the MV orifice and steered to encircle the LCT in at least one embodiment. In at least one other embodiment, the CE has a distal tip that allows it to progress directly through the valve leaflet, similar to the function of the LCT. In this latter instance, one may choose to perform the crossing of the CE in this manner so as to not modify the FE of the MV.

Referring to FIGS. 5D and 5E, the CE is placed over the LCT and then retracted proximally to engage the MV leaflets, as indicated by the directional arrow and, using mechanical and/or electrical force, the CE cuts the leaflet tissue. In one embodiment, the cut tissue remains adhered on the LCT and the CE is configured to close around the cut tissue and hold it against the LCT so as to snare the cut leaflet tissue.

Referring to FIG. 5F, in at least one other embodiment, the cut leaflet tissue is held in place by an adhesion mechanism in the form of at least one proximal portion P of the LCT, which is exposed prior to cutting. As mentioned previously, in at least one embodiment, a proximal portion P may be helical, square or rectangular, round or oval, or another geometric configuration, along with a proximal portion P that can be pre-shaped into foldable configurations. In at least one embodiment, the holding element may have a braid or adherent material that facilitates adhering the cut tissue to the LCT. In at least one embodiment, the proximal portion P may have a geometric configuration that is exposed after leaflet crossing.

The steps for use of the LCT and CE can be applied multiple times to the same MV leaflet or in different locations on the MV, including both the AML and PML, in order to modify and remove tissue as necessary. The shape and size of both the CE and LCT can be changed to tailor to the needs of the patient and subsequent therapy.

FIGS. 5G-5L show the modification and/or removal of leaflet tissue involving the MV in another embodiment of the present disclosure.

The system is delivered to the desired leaflet site with the SGC and the DC is positioned to be adjacent the AML in a manner similar to that shown in FIG. 5A.

Referring to FIG. 5G, the SGC has been withdrawn thereby exposing the DC. The LCT is then advanced out of the DC. The LCT is then used to engage and cross the AML by, for example, radiofrequency, electrical, or mechanical energy, followed by its advancement further into the LV. The LCT has a retention element in the form of a barb B at or near its tip. It also has an adhesion mechanism in form of a tensioning element TE proximal to the barb B for use in tensioning the leaflet during the procedure.

The location chosen for placement and engagement of the LCT is based on the location and the amount of leaflet tissue cutting and/or removal that is desired, as indicated from the bidirectional arrows. For example, one may choose to target relatively more tissue by positioning the LCT close to the valve annulus, or relatively less tissue by positioning the LCT close to the free edge (FE) of the leaflet. The CE can also be positioned to cross any segment of the MV as close to the FE or the MV annulus as is desired.

Referring to FIGS. 5H, the CE is then advanced through the MV orifice and steered to encircle the LCT in at least one embodiment. In at least one other embodiment, the CE has a distal tip that allows it to progress directly through the valve leaflet, similar to the function of the LCT. In this latter instance, one may choose to perform the crossing of the CE in this manner so as to not modify the FE of the MV.

Referring to FIGS. 5I and 5J, the CE is placed over the LCT and then retracted proximally to engage the MV leaflet, as indicated by the directional arrow. The tensioning element TE serves to “backstop” or hold in tension the leaflet against the CE as it is being retracted to engage the MV leaflet. In other words, the TE serves to counteract the cutting force of the LCT. Then, using mechanical and/or electrical force, the CE cuts the leaflet tissue. In one embodiment, the cut tissue remains adhered on the LCT and the CE is configured to close around the cut tissue and hold it against the LCT so as to snare the cut leaflet tissue.

Referring to FIG. 5K, in at least one other embodiment, the cut leaflet tissue is held or retained by at least the barb B of the LCT.

In yet another embodiment, a structure analogous to the tension element TE can be incorporated at the tip instead of a barb. The operation of this embodiment is analogous to the operation described with respect to previously discussed embodiments.

The steps for use of the LCT and CE can be applied multiple times to the same MV leaflet or in different locations on the MV, including both the AML and PML, in order to modify and remove tissue as necessary. The shape and size of both the CE and LCT can be changed to tailor to the needs of the patient and subsequent therapy

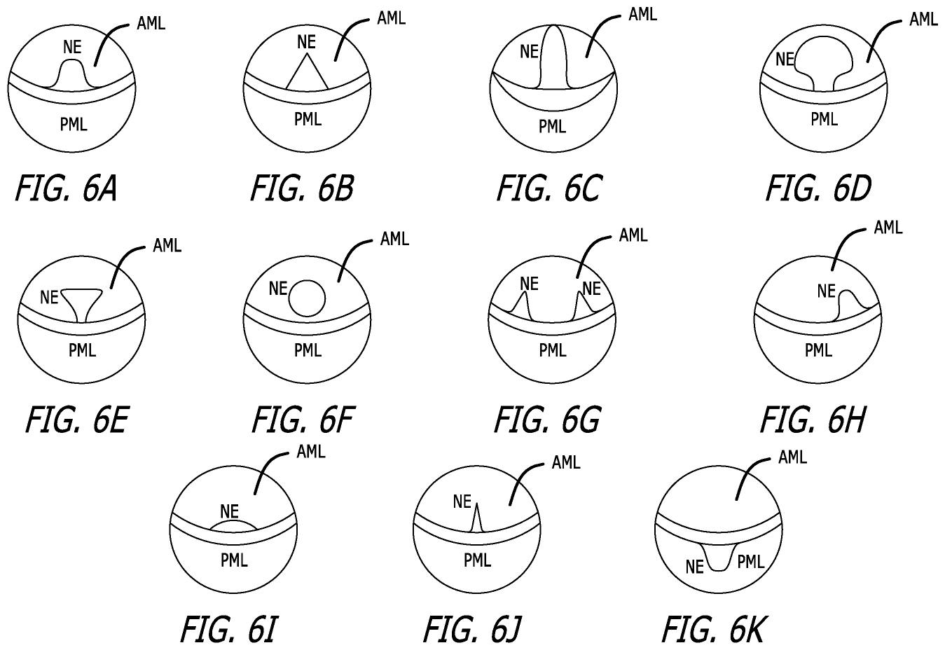

FIGS. 6A-6K show the MV following use of the present disclosure in at least a plurality of embodiments wherein the portion of the valve leaflet removed yields a newly created leaflet edge (NE). The different amounts and configurations of the leaflet tissue removed vary according to the shape and size of the CE, as well as the crossing location of the LCT.

Similar to the method used for the MV, other native heart valve leaflets and prosthetic leaflets can be modified and removed with the present invention. Heart valve leaflets and prosthetic leaflets are most commonly in tri-leaflet configurations, which is different from the MV, but the technical use of the present invention is similar regardless of the number of valve leaflets present, and regardless of whether the leaflets are native or within a prosthesis. The SGC and DC can be used to approach valve leaflets prograde or retrograde for their modification and removal with the present disclosure.

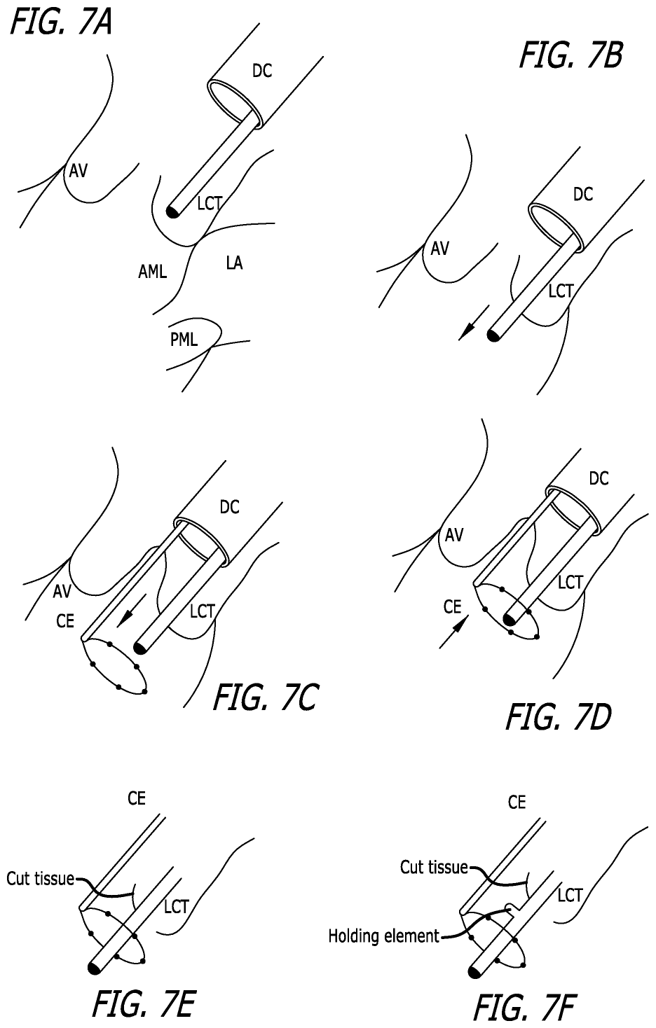

For example, FIGS. 7A-7F show the use of the present disclosure for the aortic valve, AV. Referring to FIG. 7A, following vascular access into the ascending aorta, the DC is advanced to the one of the leaflet cusps of the AV. Referring to FIG. 7B, the cutting element of the LCT is used to engage and cross the AV leaflet, for example, by electrical or mechanical energy, followed by its advancement further into the LV. As described above for MV, the location of the placement of the LCT is based on the location and the amount of leaflet tissue cutting and/or removal that is desired. For example, a user may choose to target relatively more tissue by positioning the LCT close to the aortic annulus, or relatively less tissue by positioning the LCT close to the FE. The LCT can also be positioned to cross any segment of the AV (i.e., L, R, or N cusps) as close to the FE or the AV annulus as one desires.

Referring to FIG. 7C, The CE is then advanced through the AV orifice and steered to encircle the LCT in at least one embodiment. In at least one other embodiment, the LCT has a distal tip that allows it directly through the valve leaflet, similar to the function of the CE (using mechanical or electrical energy). In this latter instance, one may choose to perform the crossing of the LCT there to preserve the native FE of the AV. The CE could be delivered across the AV in its own sheath or in the deployed state.

Referring to FIG. 7D, The CE is placed over the LCT and then retracted proximally to engage the AV leaflet, as indicated by the directional arrow.

Referring to FIG. 7E, using, for example, mechanical and/or electrical force, the CE cuts the leaflet tissue. In one embodiment, the cut tissue remains adhered to the LCT, to which the CE closes as a snare to hold the cut leaflet tissue.

Referring to 7F, in at least one other embodiment, the cut leaflet tissue is held in place by at least one proximal element P of the LCT, which, in one embodiment, is exposed prior to cutting (as mentioned above, the proximal element P may be useful during the cutting process to aid in applying counter force). As mentioned previously, at least one embodiment, such holding elements P may be helical, square or rectangular, round or oval, or another geometric configuration, along with the ability to be pre-shaped into foldable configurations. The holding element P may have a braid or adherent material that facilitates adhering the cut tissue to the LCT.

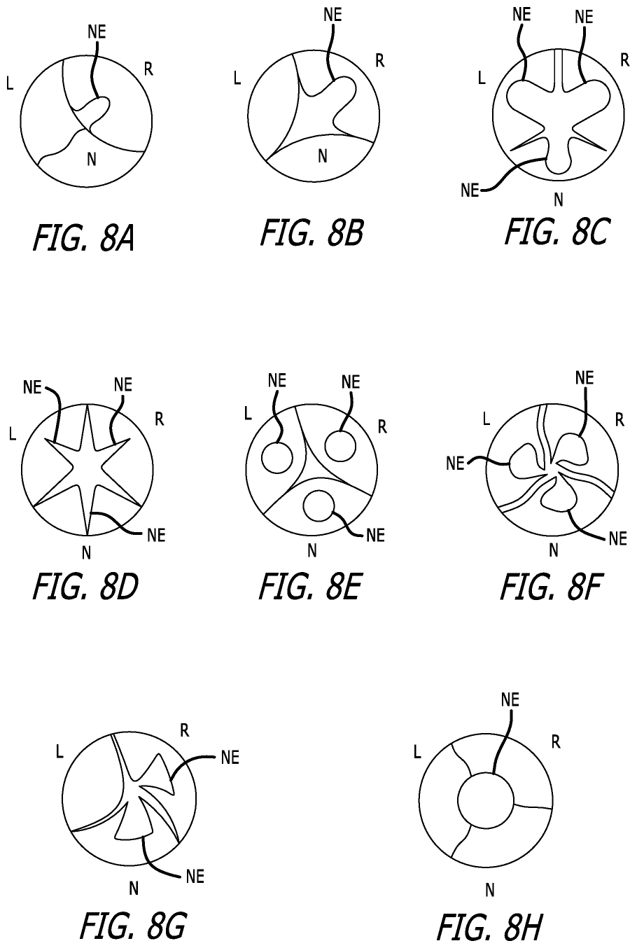

Referring to FIGS. 8A-8H, embodiments of resulting leaflet configuration of the AV are shown with the portion of the valve leaflet removed yielding a new leaflet edge (NE). The location, size of cut and amount of tissue removed varies according to the location of the LCT and configuration of the CE. The present invention can be used to treat multiple locations on the AV.

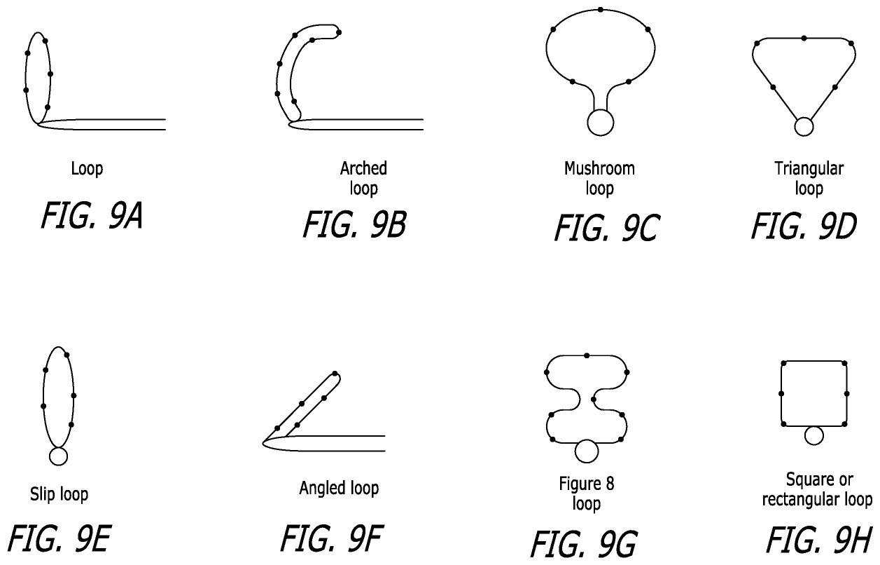

Referring to FIGS. 9A-9H, various embodiments of the CE that can be used to cut the leaflet tissue are shown. Cutting features on the CE are indicated by dots on the wire portion of the CE. In at least one embodiment, the CE has multiple cutting components that can be positioned according to the desired cut configuration.

Multiple types of cutting configurations of the CE are contemplated, including those disclosed in U.S. Publication No. 2022/0265311 entitled Method and Apparatus for Removing Heart Valve Therapy, which is incorporated herein by reference it its entirety.

In at least one embodiment, mechanical suction can be applied to hold the cut leaflet in place while being removed from the body.

In at least one embodiment, a capture basket can be deployed distal or proximal to assist in holding the cut leaflet in place while being removed from the body. In at least one embodiment, the basket facilitates entry of the cut leaflet tissue into the delivery catheters for removal. Basket configurations usable in this fashion are disclosed in U.S. Publication No. 2022/0265311 entitled Method and Apparatus for Removing Heart Valve Therapy, which is incorporated herein by reference it its entirety.

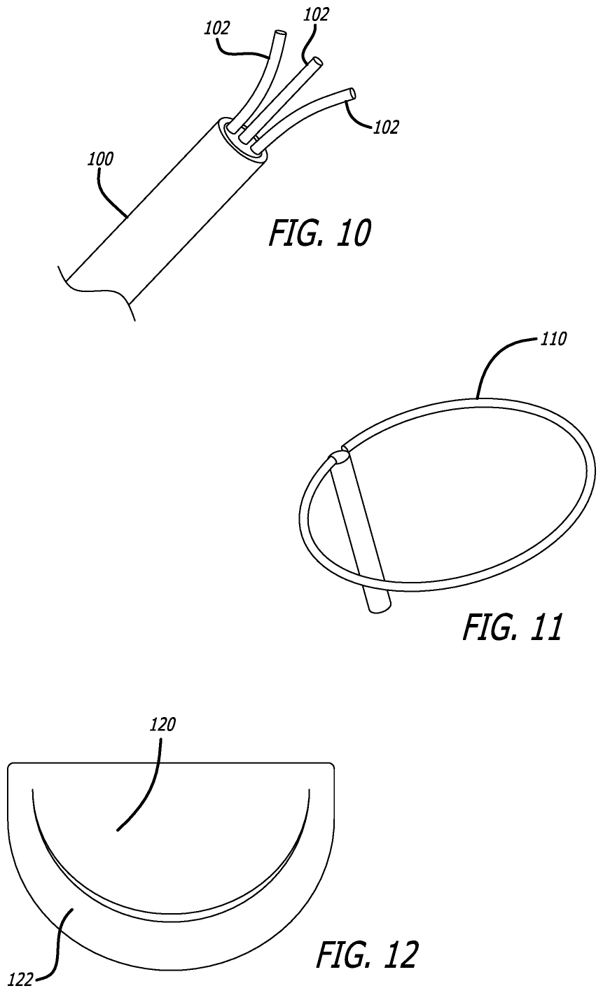

Referring to FIG. 10, an additional embodiment of an LCT 100 of present disclosure is disclosed. The LCT 100, in one embodiment, has three extensions 102 that are used to penetrate and cross the leaflet tissue. In one embodiment, the tips of the extensions include electrodes for applying cutting energy, e.g., RF energy, to the leaflet tissue.

Referring to FIG. 11, an additional embodiment of a CE 110 of the present disclosure is shown. The CE 110 has a loop that is capable of cutting tissue in a manner similar to previously disclosed embodiment of the CE. In one embodiment, the CE 110 includes electrodes for applying electrical cutting energy to the leaflet tissue.

Referring to FIG. 12, a rendering of a mitral valve MV is shown having an anterior mitral leaflet 120 and a posterior mitral valve leaflet 122.

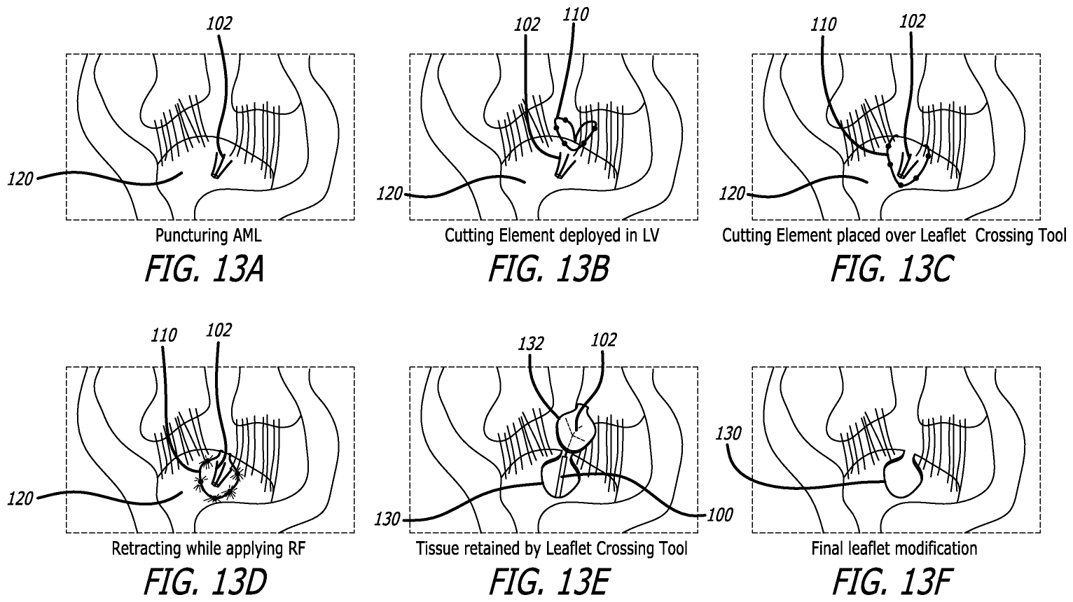

Referring to FIGS. 13A-13F, a method according to the present disclosure is shown, wherein FIG. 13A shows the extensions 102 of the LCT 100 having punctured or penetrated through the anterior mitral valve leaflet 120.

Referring to FIG. 13B, the CE 110 has been extended through the mitral valve opening into the left ventricle and directed toward the extensions 102 of the LCT 100.

Referring to FIG. 13C, the CE 110 has been secured around the extensions 102 and the electrodes placed into contact with the anterior mitral valve leaflet tissue 120.

Referring to FIG. 13D, the electrodes of the CE 110 are activated to cause a cutting action on the tissue of the anterior mitral valve leaflet 120.

Referring to FIG. 13E, the CE 110 has been withdrawn and the cut tissue 132 has been excised from the anterior mitral valve leaflet 120 and is retained on or near the extensions 102 of the LCT 100.

Referring to FIG. 13F, the final leaflet configuration is depicted after withdrawal of the LCT 100.

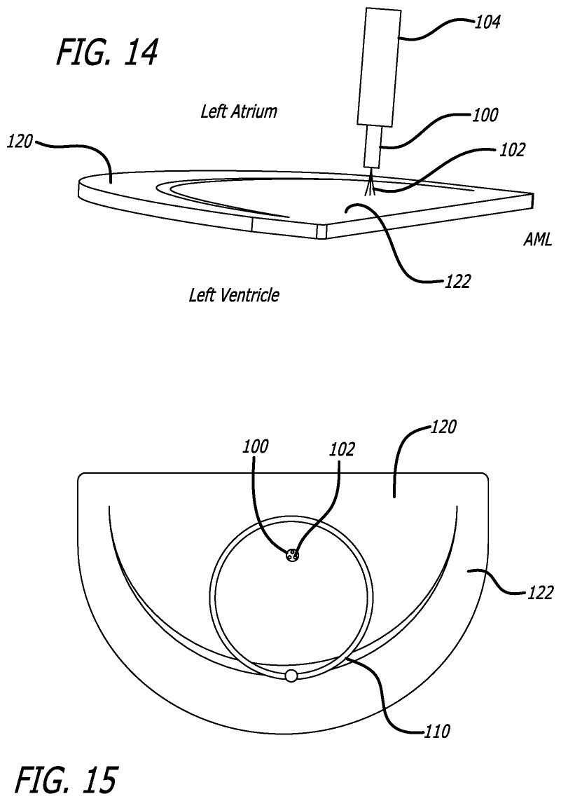

Referring to FIGS. 14 and 15, renderings are shown of the operations taking place as shown in FIGS. 13A and 13C, respectively.

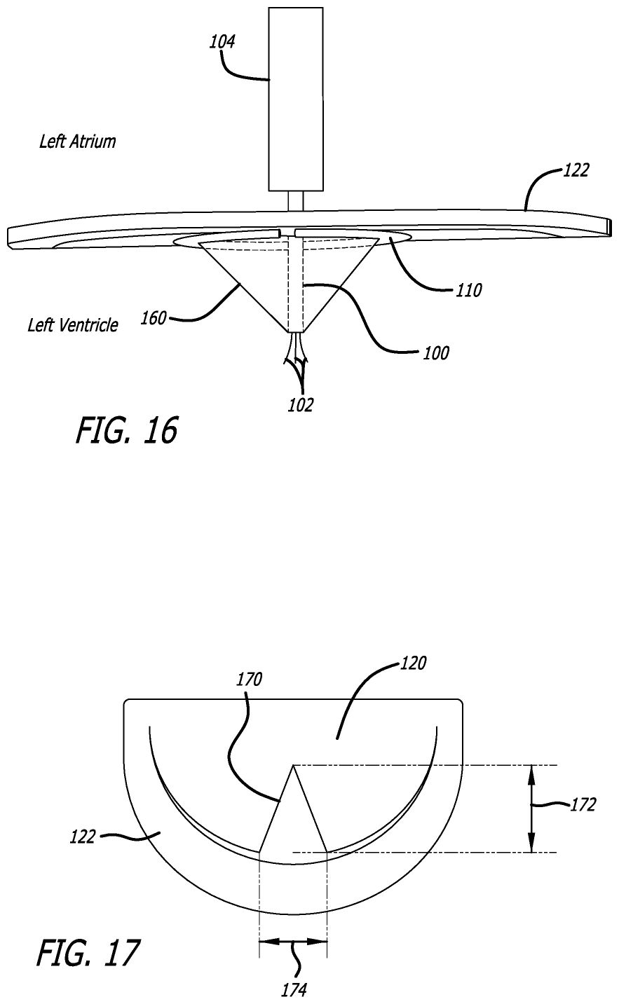

Referring to FIG. 16, another embodiment of a method according to the present disclosure is shown. Following penetration of the extensions 102 through tissue of the anterior mitral valve leaflet 120, the LCT 100 is further advanced so as to stretch and elongate the tissue of the anterior mitral valve leaflet into a conical or “teepee” like shape. Such an action serves to create a tension or tautness in the tissue so as to facilitate the cutting action of the CE 110.

Referring to FIG. 17, an embodiment of the leaflet modification achieved is shown as having a triangular shape with a leaflet modification length 172 and a leaflet modification width 174. According to desired placement of the LCT 100 and the configuration of the CE 110, there are many different locations and shapes of leaflet modifications that can be achieved as described in other embodiments of the present disclosure.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method. article, or apparatus that comprises a list of elements is not necessarily limited to only those elements but may include other elements not expressly listed or inherent to such process, method, article, or apparatus.

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Still further, the figures depict preferred embodiments for purposes of illustration only. One skilled in the art will readily recognize from the discussion herein that alternative embodiments of the structures and methods illustrated herein may be employed without departing from the principles described herein.

Upon reading this disclosure, those skilled in the art will appreciate still additional alternative structural and functional designs. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1-15. (canceled)

16. A system of modifying a heart valve leaflet, comprising:
a delivery catheter;
a leaflet crossing tool comprising a crossing element located at a distal tip of a needle or elongated segment; the crossing element providing mechanical, radiofrequency, and/or electrical energy for puncturing a heart valve leaflet; the leaflet crossing tool advanced out of the delivery catheter in an operational configuration of the system; and,
a cutting element comprising a wire portion in a shape of a loop adapted for applying electrical cutting energy; the cutting element advanced out of the delivery catheter in the operational configuration of the system;
wherein, in the operational configuration of the system, the loop of the cutting element is positioned around the cutting element and is retractable proximally relative to the cutting element.

17. The system of claim 16, wherein the needle or elongated segment is straight, helical, square, rectangular, round, oval, or bent.

18. The system of claim 17, wherein a proximal portion of the needle or elongated segment comprises a pre-shaped foldable configuration.

19. The system of claim 18, wherein the pre-shaped foldable configuration comprises a single notch or multiple notches.

20. The system of claim 16, wherein the crossing element comprises three extensions that each include electrodes for applying cutting energy.

21. The system of claim 16, wherein the wire portion comprises a plurality of electrodes for applying cutting energy.

22. The system of claim 16, wherein the loop of the wire portion comprises a circular loop shape, arched loop shape, mushroom loop shape, triangular loop shape, slip loop shape, angled loop shape, FIG. 8 loop shape, square loop shape, or rectangular loop shape.

23. The system of claim 16, further comprising a tensioning element in a form of an enlarged backstop on the leaflet crossing tool.

24. The system of claim 23, wherein the leaflet crossing tool further comprises a barb located at or near the distal tip.

25. The system of claim 16, wherein the needle or elongated segment is straight, helical, square, rectangular, round, oval, or bent; wherein the wire portion comprises a plurality of electrodes for applying cutting energy; wherein the loop of the wire portion comprises a circular loop shape, arched loop shape, mushroom loop shape, triangular loop shape, slip loop shape, angled loop shape, FIG. 8 loop shape, square loop shape, or rectangular loop shape; and further comprising a tensioning element in a form of an enlarged backstop on the leaflet crossing tool.
